(19) United States
(12) Patent Application Publication
(10) Pub. No.: US 2017/0021093 A1
(43) Pub. Date: Jan. 26, 2017

(54) MEDICAL DEVICE WITH SELF-SUSTAINING POWER SOURCE

(71) Applicant: LifeScan Scotland Limited, Inverness (GB)

(72) Inventors: David ELDER, Inverness (GB); Malcolm D. HAMER, Inverness (GB)

(21) Appl. No.: 14/808,127

(22) Filed: Jul. 24, 2015

(51) Int. Cl. A61M 5/145; H03K 21/02; H01L 41/113; A61M 5/142; H01L 41/04

(52) U.S. Cl. CPC A61M 5/1454; A61M 5/14248; H01L 41/042; H01L 41/113; H03K 21/023; A61M 2005/14208; A61M 2005/14506; A61M 2210/04; A61M 2205/52; A61M 2205/825

(57) ABSTRACT

A medical device with a self-sustaining power source is disclosed herein. The medical device includes a pump and at least one mechanical activation mechanism for engaging the pump to cause a dose event. An energy generator coupled to the activation mechanism generates energy each time the activation mechanism is actuated. The generated energy is supplied to a dose counter of the infusion device.

### TECHNICAL FIELD

This application generally relates to the field of medical devices and more specifically to a mechanically operated medical device, such as a portable infusion pump, that includes a power source that employs energy harvesting.

### BACKGROUND

Tight control over the delivery of insulin in both type I diabetes (usually juvenile onset) and type II diabetes (usually late adult onset), has been shown to improve the quality of life as well as the general health of these patients. Insulin delivery has been dominated by subcutaneous injections of both long acting insulin to cover the basal needs of the patient and by short acting insulin to compensate for meals and snacks. Recently, the development of electronic, external insulin infusion pumps has allowed the continuous infusion of fast acting insulin for the maintenance of the basal needs as well as the compensatory doses (boluses) for meals and snacks. These infusion systems have shown to improve control of blood glucose levels. However, they suffer the drawbacks of size, cost, and complexity. For example, these pumps are electronically controlled and must be programmed to supply the desired amounts of basal and bolus insulin. This prevents many patients from accepting this technology over the standard subcutaneous injections.

Thus, a number of highly compact mechanical solutions, such as the Calibra Finesse© insulin patch pump, have been create to provide a convenient form of insulin treatment which does not require significant programming or technical skills to implement to service both basal and bolus needs has been developed. Such an infusion device is simple to use and mechanically driven, negating the need for batteries and the like. The infusion device can be directly attached to the body and does not require any electronics to program the delivery rates. The insulin is preferably delivered through a small, thin-walled tubing (cannula) through the skin into the subcutaneous tissue similar to technologies in the prior art.

Historical information indicating when a patient received a dose is important in managing chronic conditions and diseases, such as diabetes. Insulin-dependent diabetics, for example, need to know how much insulin they have injected into their body and when, so that they can determine how much insulin they should receive to compensate for meals, etc. However, because these compact insulin delivery devices are purely mechanical, there is no way of storing dosing information. The addition of electronics can provide a way to store dosing information, but the electronics require a power source. Adding a power source can increase the size of the device, as well as rendering disposal of the device in accordance with regulations problematic. Further, traditional power sources, such as batteries, may require a method of charging the device that will increase the cost and complexity of the device. A method of adding batteryless near-range wireless capability has been proposed. However, this method does not include a way of adding a timestamp to the dosing information.

### BRIEF DESCRIPTION

Various embodiments of a power source for a mechanical medical infusion device are described herein. Advantageously, this power source is self-sustaining and provides a technique for adding a timestamp to dosing information. Further, the medical infusion device, including this power source, can be disposed of following use and in accordance with prescribed regulations without undue burden.

In a first aspect, an infusion device is described. The infusion device includes a housing having a reservoir that is sized to retain a quantity of liquid medicament and a mechanical pump that displaces a portion of the liquid medicament when actuated mechanically such as, for example by the muscles of a user. A mechanically driven activation mechanism is disposed on the housing for actuating the pump in order to create a dose event. An energy generator is coupled to the activation mechanism. The energy generator is configured to generate energy upon each operation of the mechanically driven activation mechanism in order to power a dose counter that is configured to record dose events.

According to another aspect, a power source for a medical infusion device is described. The infusion device includes a pump and at least one mechanical activation mechanism for engaging the pump to cause a dose event. The power source can include at least one piezo crystal physically connected to the activation mechanism. The at least one piezo crystal is configured to generate a predetermined amount of energy when the infusion device is activated.

According to yet another aspect, a method for providing electrical power to an infusion device is described. The infusion device includes a pump and at least one mechanically operated activation mechanism for engaging the pump to cause a dose event. The method includes generating a predetermined amount of energy each time the mechanically operated activation mechanism of the infusion device is engaged and using the generated energy to power a dose counter of the infusion device.

In addition to the various aspects described above, other features recited below can be utilized in conjunction therewith to arrive at different permutations of the invention. For example, the energy generator may include at least one piezo crystal coupled to the activation mechanism, the at least one piezo crystal being configured to produce a predetermined amount of energy when the activation mechanism is actuated; the device may further include an energy storage device configured to store energy generated by the energy generator; the energy storage device may include at least one capacitor; the dose counter may include a microcontroller and wherein the energy storage device is coupled to the microcontroller; the activation mechanism may include at least one depressible button coupled to the pump wherein at least one said piezo crystal is coupled to the at least one depressible button; the activation mechanism is configured to toggle between a non-actuated position and an actuated position and wherein the piezo crystal produces piezo energy pulses upon toggling of the activation mechanism between the non-actuated position and the actuated position; the piezo crystal is cantilevered to the activation mechanism such that flexure of the piezo crystal occurs upon toggling of the activation mechanism between the non-actuated position and the actuated position; the piezo energy pulses produced by the flexure of the piezo crystal have opposite polarities based on the toggled position of the activation mechanism; the device may further include circuitry configured to convert the piezo pulses to a common polarity; the device may further include circuitry for conditioning the piezo energy pulse produced by the piezo crystal; the circuitry further may include an oscillating crystal that is configured to provide a timing signal based on a generated piezo pulse; the dose counter is configured to provide a time stamp of the dose event based on the timing signal; the device may further include a communication interface for communicating with another device; the communication interface may include a near field communication (NFC) interface; operation of the communication interface by said other device generates a predetermined amount of energy and wherein the power source further harvests the generated predetermined amount of energy; the harvested energy is stored in the energy storage device and the predetermined amount of energy includes approximately 80 microJoules; the harvested energy is used to initiate the dose counter; the device is configured to administer insulin to a patient; the device may include a portable housing that is configured to be attached directly to skin of a patient.

One advantage realized is that a compact and mechanically operated infusion device can be configured to include a self-sustaining power source, using energy harvesting. This power source can enable enhanced features, such as dose counting, recording, and transmission, to be realized. A further advantage is the ability to include a timestamp when recording the dose counting.

Another advantage is that the inclusion of an energy generator as described herein does not significantly impact manufacturability or footprint of the infusion device, enabling the same to remain compact and portable.

These and other features and advantages will become apparent to those skilled in the art when taken with reference to the following more detailed description of the exemplary embodiments of the invention in conjunction with the accompanying drawings that are first briefly described.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention (wherein like numerals represent like elements).

FIG. 1 is a perspective view of a mechanically operated infusion device;

FIG. 2 is a schematic representation of the valves and pump of the infusion device of FIG. 1;

FIG. 3 is an exploded assembly view of an infusion device in accordance with an exemplary embodiment;

FIG. 4 is a partial functional view of an infusion device having an energy generator according to an exemplary embodiment including activation mechanisms of the infusion device in a first position;

FIG. 5 is a partial functional view of the infusion device of FIG. 4 with the activation mechanisms in a second or engaged position to enable energy pulses to be generated;

FIG. 6 is a functional block diagram of a dose counter powered by the power source of the infusion device; and

FIG. 7 is a flowchart depicting an exemplary method of providing power to an infusion device.

### DETAILED DESCRIPTION

The following detailed description should be read with reference to the drawings, in which like elements in different drawings are identically numbered. The drawings, which are not necessarily to scale, depict selected embodiments and are not intended to limit the intended scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “patient” or “user” refer to any human or animal subject and are not intended to limit the systems or methods to human use, although use of the subject invention in a human patient represents a preferred embodiment.

The term “medicament” means a volume of a liquid, solution or suspension, intended to be administered to a patient. As used herein, the terms “comprising” , “comprise” and “comprises” are open-ended terms intended not to be fully inclusive and in which the terms “include”, “including” and “includes” are intended to have the same intent. While the device(s) are herein described as having “one” part or component, it is to be understood that the term “one” implicitly refers to “at least one”.

The terms “about” and “substantially” are used in connection with a numerical value throughout the description and claims denote an interval of accuracy, familiar and acceptable to a person skilled in the art. The interval governing this term is preferably ±20%. Unless specified, the terms described above are not intended to narrow the scope of the invention as described herein and according to the claims.

Certain exemplary embodiments will now be described to provide an overall understanding of the principles of the structure, function, manufacture, and use of the systems and methods disclosed herein. One or more examples of these embodiments are illustrated in the accompanying drawings. Those skilled in the art will understand that the systems and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure.

As will be discussed in more detail below, the disclosed systems and methods relate to a mechanically operated medical infusion device having a pump and at least one activation mechanism, such as a depressible button, for engaging the pump to cause a dose event. A power source is provided for the device that includes at least one energy generator connected to the activation mechanism and configured to generate a predetermined amount of energy when the mechanism is activated.

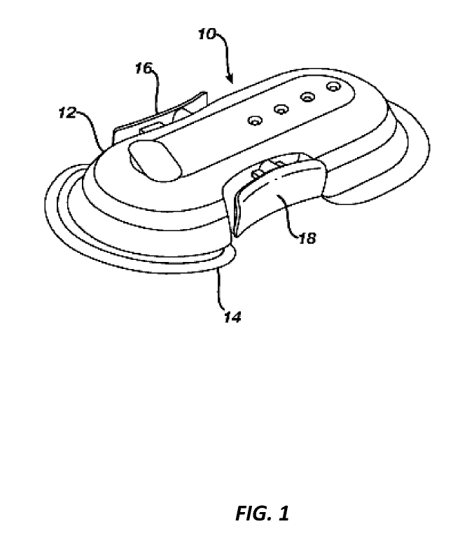

FIG. 1 depicts a perspective view of an infusion device. The infusion device 10 generally includes an enclosure 12, a base 14, a first activation mechanism 16, and a second activation mechanism 18. In an example, illustrated here, the first activation mechanism 16 and the second activation mechanism 18 are depressible buttons disposed on opposing sides of the enclosure 12. The activation mechanisms 16, 18 are each configured to toggle between a first, non-actuated position and a second, actuated position. It is to be understood that while the infusion device 10 is illustrated herein as including two activation buttons, the infusion device 10 can include at least one activation mechanism.

According to this version, the enclosure 12, as will be seen subsequently, is formed by a series of multiple device layers being brought together. Each device layer defines various components of the device 10 such as, for example, a reservoir, various fluid conduits, pump chambers, and valve chambers. This form of device construction, in accordance with aspects of the present invention, enables manufacturing economy to an extent rendering the device disposable after intended use by a patient.

The base 14 preferably includes an adhesive coating (not shown) to permit the device 10 to be adhered to a patient's skin. The adhesive coating may originally be covered with a releasable cover (not shown) that may be peeled from the base 14 when the patient endeavors to deploy the device 10 and attach the device 10 to the skin of the patient. Such arrangements are well known in the art.

The infusion device 10 may be mated with a previously deployed cannula assembly. However, it is contemplated herein that the various aspects of the present invention may be realized within a device that may be alternatively first adhered to the patient's skin followed by the deployment of a cannula thereafter.

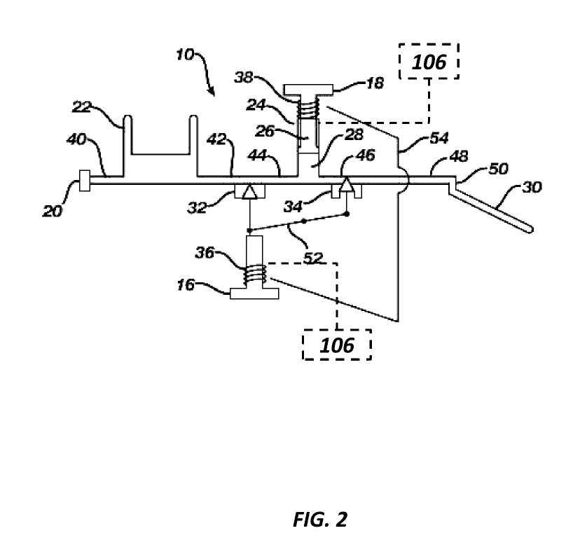

As noted, the activation mechanisms 16 and 18 are placed on opposite sides of the device 10 and directly across from each other. This positioning more readily insures the concurrent depression of the buttons when the patient wishes to receive a dose (bolus) of the liquid medicament contained within the device 10. This arrangement also imposes substantially equal and opposite forces on the device 10 during dosage delivery to prevent the device 10 from being displaced and possibly stripped from the patient. As will be further seen hereinafter, the concurrent depression of the buttons 16, 18 is used to particular advantage. More specifically, the first activation mechanism 16 may serve as a valve control which, when in a first position as shown in FIG. 2, establishes a first fluid path between the device reservoir and the device pump to support pump filling, and then, when in a second or depressed position, establishes a second fluid path between the device pump and the device outlet or cannula to permit dosage delivery to the patient. In addition, a linkage between the control activation mechanisms 16 and 18 permits actuation of the device pump with the second activation mechanism 18 only when the second fluid path has been established by the first activation mechanism 16. Hence, the first activation mechanism 16 may be considered a safety control. Addition details regarding the features of the exemplary infusion device can be found in pending U.S. patent application Ser. No. 14/289,930, entitled “Manually Actuated Infusion Device and Dose Counter,” published as U.S. Patent Application Publication No. 2014/0378903A1, and in U.S. Pat. No. 7,976,500, entitled “Disposable Infusion Device with Redundant Valved Safety,” the entirety of which are incorporated by reference. As will be further described by the following figures, the infusion device 10 further includes an energy generator 106 that is coupled to the activation mechanisms 16, 18.

FIG. 2 provides a schematic representation of the valves and pump of the infusion device 10 of FIG. 1. More specifically, the infusion device 10 further includes a fill port 20, a reservoir 22, a pump 24, and the cannula 30. The infusion device 10 further includes a first valve 32 and a second valve 34. A plurality of fluid conduits are provided. More specifically and according to this version, a fluid conduit 40 provides a fluidic connection between the fill port 20 and the reservoir 22, fluid conduit 42 provides a fluidic connection between the reservoir 22 and the first valve 32, fluid conduit 44 provides a fluidic connection between the first valve 32 and the pump 24, fluid conduit 46 provides a fluidic connection between the pump 24 and the second valve 34, and fluid conduit 48 provides a fluidic connection between the second valve 34 and the device outlet 50. The outlet 50 is arranged to communicate with the cannula 30.

It may also be noted that the activation mechanisms 16 and 18 are spring-loaded or biased by springs 36 and 38. The springs 36, 38 are provided for returning the activation mechanisms 16, 18 to the first position after a bolus of the contained fluid medicament is administered.

The pump 24 of the infusion device 10 comprises a piston pump 24. The pump 24 includes a pump piston 26 and a pump chamber 28. In accordance with this embodiment, the activation mechanism 18 is directly coupled to and is an extension of the pump piston 26.

With further reference to FIG. 2, the device 10 additionally includes a first linkage 52 and a second linkage 54. The first linkage 52 is a toggle linkage between the first valve 32 and the second valve 34. The first linkage 52 is arranged to assure that the second valve 34 does not open until after the first valve 32 is closed. The second linkage 54 is provided between the first activation mechanism 16 and the second activation mechanism 18. The second linkage 54 is arranged to assure that the pump 24 does not pump until after the first valve 32 is closed and the second valve 34 is opened by the first activation mechanism 16.

Still further, the second valve 34 is a safety valve that closes tighter responsive to increased fluid pressure within the fluid conduit 46. This closure assures that liquid medicament is not accidentally administered to the patient notwithstanding the inadvertent application of pressure to the reservoir 22, for example. In applications such as this, it is not uncommon for the reservoir 22 to be formed from a flexible material. While this manufacture has certain advantages, it does present the risk that the reservoir 22 may be accidentally squeezed as it is worn by the patient. Because the second valve 34 only closes tighter under such conditions, it is assured that increased accidental reservoir pressure will not cause the fluid medicament to flow to the cannula 30.

In operation, the reservoir 22 is first filled through the fill port 20 to a desired level of medicament. In this state, the first and second valves 32 and 34 will be in the positions as shown in which the first valve 32 is open and the second valve 34 is closed. This configuration permits the pump chamber 28 to be filled after the reservoir 22 is filled. The cannula 30 may then be deployed followed by the deployment of the infusion device 10. In this state, the first and second valves 32 and 34 will remain in the depicted configuration with the first valve 32 being open and the second valve 34 closed. This arrangement permits the pump chamber 28 to be filled through a first fluid path, including conduits 42 and 44, as the piston 26 returns to its first position after each applied dose.

When the patient wishes to receive a dose of medicament, the opposing activation mechanisms 16, 18 are concurrently pressed using mechanical power of the patient's fingers. As used herein, the term “mechanically driven” or “mechanically actuated” indicates that the primary power source is muscle in nature. According to this version of the device 10, the first linkage 52 causes the first valve 32 to close and the second valve 34 to thereafter open. Meanwhile, the second linkage 54 precludes actuation of the pump 24 until the first valve 32 is closed and the second valve 34 is opened by the first activation mechanism 16. At this point, a second fluid path is established from the pump 24 to the cannula 30 through fluid conduits 46 and 48 as well as the outlet 50. The medicament is then administered to the patient through the cannula 30.

Once the medication dosage is administered, the piston 26, and thus the activation mechanism 18, is returned under the biasing pressure of the spring 38 to its initial position. During the travel of the piston 26 back to its first position, a given volume of the liquid medicament for the next dosage delivery is drawn from the reservoir 22 into the pump chamber 28 to ready the infusion device 10 its next dosage delivery.

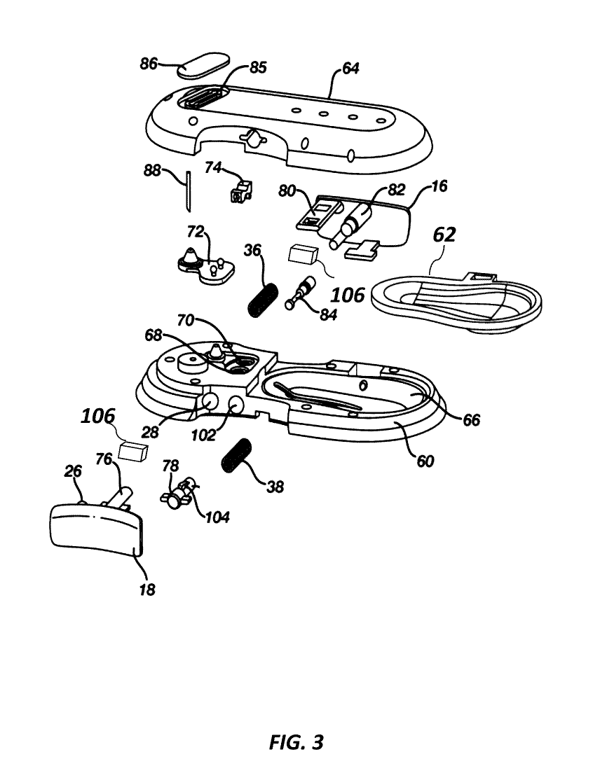

FIG. 3 is an exploded assembly view of the infusion device 10 of FIGS. 1 and 2. The main component parts include the aforementioned device layers including a base layer 60, a reservoir membrane or intermediate layer 62, and a top body layer 64. The base layer 60 is a substantially rigid unitary structure that defines a first reservoir portion 66, the pump chamber 28, and valve sockets 68 and 70 of the first and second valves 32, 34, FIG. 2, respectively. The base layer 60 may be formed of plastic, for example. The reservoir membrane layer 62 is received over the reservoir portion 66 to form the reservoir 22, FIG. 2. A valve seat structure 72 is received over the valve sockets 68 and 70 to form the first and second valves 32 and 34, FIG. 2, respectively. A rocker 74 is placed over the valve seat structure 72 in order to open and close the valves 32, 34 as will be seen subsequently. The second or pump activation mechanism 18 carries the pump piston 26 that is received within the pump chamber 28. The pump activation mechanism 18 also carries a cam cylinder 76 with a lock tube 78 therein that form a portion of the second linkage 54, FIG. 2. The spring 38 returns the second activation mechanism 18 to its first position after each dosage delivery.

The first activation mechanism 16 carries a valve timing cam 80 that rocks the rocker 74. The mechanism 16 further carries a cam cylinder 82 and a cam pin 84 that is received into the cam cylinder 82. The spring 36 returns the activation mechanism 16 to its first position after each dosage delivery. The top body layer 64 forms the top portion of the device enclosure. This layer 64 receives a planar cap 86 that completes fluid paths 85 partially formed in the top layer 64. Lastly, a needle 88 is provided that provides fluid coupling from the cannula 30, FIG. 2, to the outlet 50, FIG. 2, of the device 10.

According to this exemplary embodiment, each activation mechanism 16, 18 includes an energy generator 106, shown schematically in FIG. 3, that is coupled to the activation mechanisms 16, 18. Upon actuation of each of the activation mechanisms 16, 18 in the manner previously described, the energy generators 106 are configured to generate a predetermined amount of energy. As will be discussed further below, the energy generated by the energy generators 106 is used to power various components of the infusion device 10, such as a dose counter as subsequently described.

As previously described, the infusion device 10 described herein is capable of delivering discrete doses or boluses of medication to the patient based on engagement of the activation mechanisms 16 and 18. Most, if not all, patients may desire a way for their infusion device to record when a dose is delivered in a dose event. Thus, as will be further discussed below, the infusion system 10 can include a dose counter (not shown) to record dose events.

It has been determined by applicant that transmitting the occurrence of each dose to a remote device such as a mobile device (e.g., a smartphone, a tablet PC, etc.) is desirable, as the structure and method for doing so minimizes the number of components that need to be added to the infusion device of FIGS. 1-3. A near-field communication (NFC) interface, for example, can be used to locally transmit the occurrence of each dose.

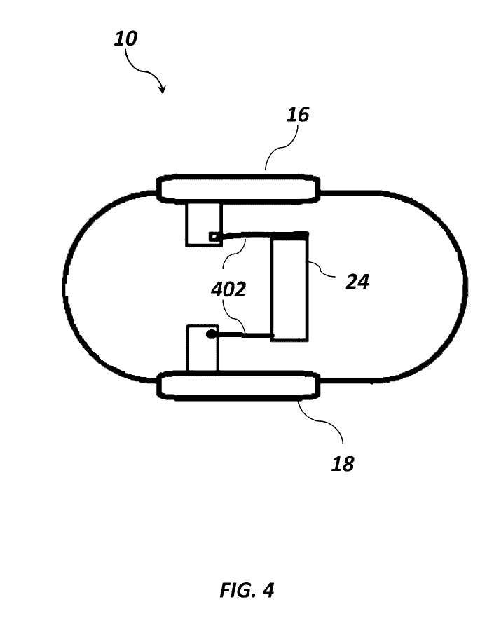

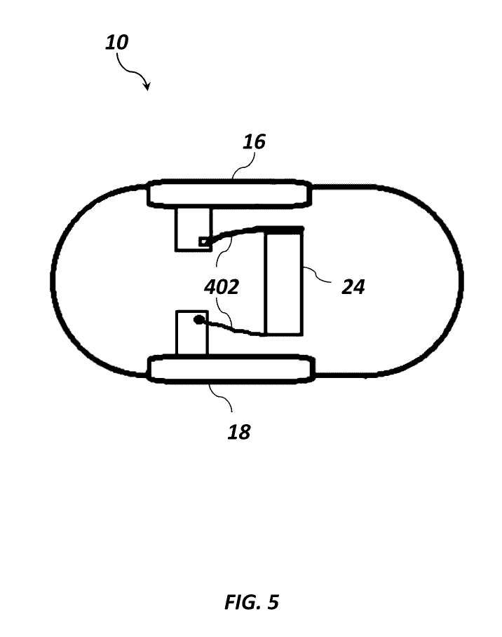

Referring now to FIGS. 4-5, depicted is a functional view of the infusion device 10 (partially shown), including a power source for the dose counter. As previously discussed, the infusion device 10 includes a first activation mechanism 16 and a second activation mechanism 18 disposed on opposite sides. As described previously with regard to FIGS. 2-3, the concurrent operation of the activation mechanisms 16, 18 activates a pump 24 to dispense a dose of medication as a dose event. The activation mechanisms 16, 18 toggle between a first, non-activated position, illustrated in FIG. 4, and a second, activated position, illustrated in FIG. 5.

As illustrated in FIGS. 3 and 4, the energy generator 106 is mechanically coupled to each activation mechanism 16, 18. In addition, the end opposite the end coupled to the activation mechanism 16, 18 can be mechanically coupled, for example, to a fixed portion of the pump 24, as illustrated in FIGS. 4-5. In an embodiment illustrated herein, the energy generators 106 are piezo crystals 402, such as lead zinconate titanate crystals or sodium potassium niobate crystals, among others. The piezo crystals 402 can be fixed using electromechanical means, i.e., any mechanical fixing that also allows the two opposing sides of each piezo crystal 402 to be electrically connected. These electromechanical means include clamping, gluing with a conductive glue, and using threaded or similar fasteners using a hole (not shown) formed in the piezo crystal 402, among others. As noted and in this embodiment, a piezo crystal 402 is coupled to each activation mechanism 16, 18 and to the pump 24. According to this embodiment, the piezo crystals 402 are coupled to the activation mechanisms 16, 18 in a cantilevered fashion. The piezo crystals 402 can have any suitable size and shape. The size and shape of the piezo crystals 402 can be determined by the mechanical constraints of fitting the piezo crystals 402 inside the infusion device 10. For example, each piezo crystal 402 can have a rectangular shape having a dimension of 5×10 mm. To preserve useful life, care is taken not to overflex the piezo crystals 402 during operation.

In this embodiment, the piezo crystal(s) 402 are mechanically attached at one end into the body of the infusion device 10. The opposite end(s) of the piezo crystals 402 need not be attached to the activation mechanisms 16, 18, although they can be. The main need is that the piezo crystals 402 flex upon actuation of the pump 24. Alternatively, the activation mechanisms 16, 18 can have a serrated edge in contact with an edge of the piezo crystal(s) 402. In this embodiment, depression of each button 16, 18 causes the serrated edge of the button 16, 18 to move across the end of the corresponding piezo crystal 402, giving the crystal 402 several ‘pings’ on both actuation and relaxation motions. In this way, crystal flexure is reduced and replaced with multiple dual polarity pulses for each activation stroke.

As illustrated by FIG. 4, when the activation mechanisms 16, 18 are in the first, non-activated position, the activation mechanisms 16, 18 exert no pressure on the piezo crystals 402. Thus, the piezo crystals 402 are in a non-stressed state. As illustrated by FIG. 5, when the activation mechanisms 16, 18 are each moved to a second, activated position in which the activation mechanisms 16, 18 are depressed, the activation mechanisms 16, 18 exert a pressure on the piezo crystals 402. This pressure causes the piezo crystals 402 to flex or deform. Flexing of the piezo crystals 402 between the first and second positions generates power in the form of a piezo energy pulse, which is harvested to power components of the infusion device, particularly the dose counter. Each change of the piezo crystals 402 between the flexed and non-flexed state generates a piezo energy pulse. Thus, actuation of the activation mechanism 16, 18 according to this embodiment produces a total of four (4) piezo energy pulses, as each of the activation mechanisms 16, 18 is depressed to the activated position, flexing the piezo crystals 402, and released to return to the non-activated position, allowing the piezo crystals 402 to return to a resting, non-stressed state.

The voltage of the piezo energy pulses produced by the piezo crystals 402 is dependent upon the geometry and flexure of the crystals 402. Because the piezo pulses are produced by the piezo crystals 402 moving reversibly between their flexed and non-flexed states, the generated piezo pulses have opposing polarities. As will be discussed further below, the piezo pulses are preferably converted to a common polarity. For example, the negative piezo pulses can be converted to positive piezo pulses for purposes of energy storage by the infusion device 10.

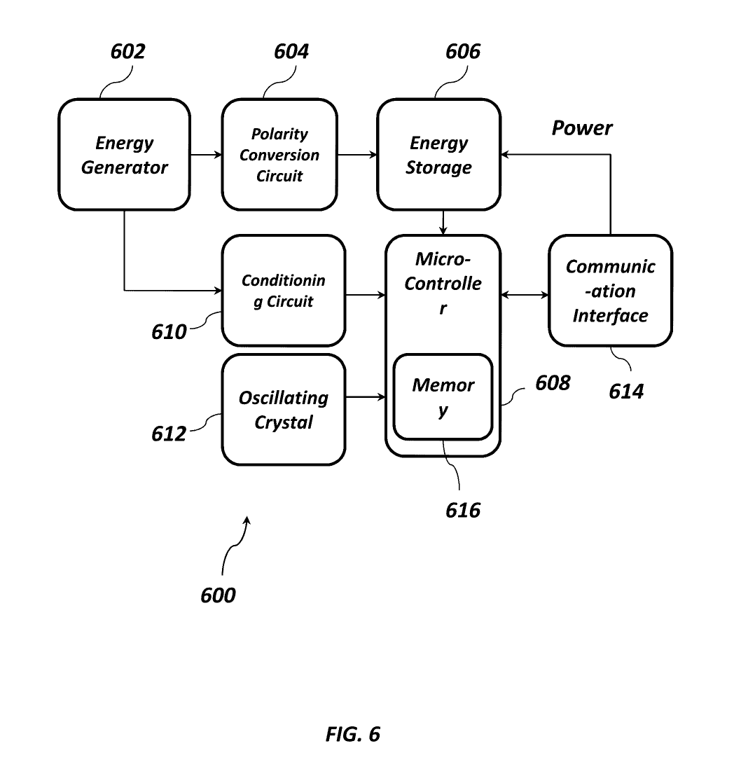

Referring now to FIG. 6, a block diagram of an exemplary dose counter 600, including the power source, is illustrated. The dose counter 600 includes an energy generator 602. As discussed above, the energy generator 602 is coupled to at least one activation mechanism of an infusion device. For example, an energy generator 602 can be coupled to each activation mechanism of the infusion device. In an embodiment, the energy generator 602 is a piezo crystal. The energy generator 602 generates energy each time the activation mechanism is actuated.

The energy produced by the energy generator 602 is stored by at least one energy storage device represented by block 606. The energy storage device 606 can include at least one capacitor configured for storing the generated energy or other suitable devices. According to the exemplary embodiment, the capacitor can be a small package capacitor, such as the AVX F750G228KRC 2200 μF tantalum capacitor in a 2824 case. The energy storage device 606 is coupled to a micro-controller 608 of the dose counter 600. The energy stored by the energy storage device 606 is provided to the micro-controller 608 to power the dose counter 600.

As discussed above, the energy pulses produced by the energy generator 602 can have opposing polarities. Converter circuitry 604 is provided that converts the opposing polarities to a single polarity. For example, a rectifier or other suitable circuitry is provided to convert negative piezo energy pulses to positive piezo pulses. According to this embodiment, a rectifier is coupled to each piezo crystal.

According to this embodiment, a real time clock 612 is coupled to the micro-controller 608. The clock 612 tracks and maintains system time. According to this embodiment, the clock 612 creates a timing signal used in conjunction with a generated piezo pulse in the case of a piezo crystal as the energy generator 602. For example, the real time clock 612 can include an oscillating crystal, such as a 32.768 kHz oscillating crystal, that is suitably linked or coupled to the micro-controller 608. Alternatively, the micro-controller 608 can maintain a software real time clock based upon as oscillation signal provided by the clock 612.

As shown in FIG. 6, pulse conditioning circuitry 610 is used to convert the energy generated by the energy generator 602 into an electrical signal, such as a digital signal. This converted electrical signal is then transmitted to the micro-controller 608. Upon receiving the electrical signal, the micro-controller 608 is programmed to record a dose event. For example, the micro-controller 608 can advance a counter to log the dose event. In another example, the micro-controller 608 can also store the occurrence of a dose record into memory 616. For purposes of storage, the memory 616 can be any suitable type of memory. For example, random-access memory (RAM) or electrically erasable programmable read-only memory (EEPROM) can be used. According to at least one version, the memory 616 can be a ferroelectric random access memory (FRAM).

Providing a timestamp of the dose event is preferred. Therefore, the micro-controller 608 is additionally configured to record the time of the dose event, as maintained by the clock 612 and the timing signal generated by the oscillating crystal with each piezo pulse. In an example, the electrical signal additionally acts as a wake-up signal. In this embodiment, the micro-controller 608 is in a resting state when not recording a dose event in order to reduce energy consumption. Upon receiving the electrical signal, the micro-controller 608 exits the resting state and records the dose event.

A communication interface 614 is coupled to the micro-controller 608. Using this communication interface 614, the micro-controller 608 transfers the recorded dose event information to another device, such as a smartphone (not shown). In an example, the micro-controller 608 transfers a dose event record each time a record is generated. In another example, the micro-controller 608 can transfer dose event records on demand when another device initiates communication with the micro-controller 608 via the communication interface 614. The communication interface 614 can be any suitable type of communication interface employing a local wireless protocol covered under relevant portions of IEEE 802.11. For example, the communication interface 614 can be a near field communication (NFC) interface or other low power wireless communication links, such as BlueTooth™, Zigbee, and ANT, among others. Alternatively, a hard-wired connection could be provided between the infusion device and the other device.

In an embodiment, operation of the communication interface 614 generates energy. This energy is harvested and stored in energy storage device 606. Thus, this energy harvested from the communication interface 614 initially powers the micro-controller 608. In an embodiment, when the reservoir of the infusion device is initially filled and attached to a patient, the patient scans the infusion device with another device, such as a mobile device, to initially configure the device using the communication interface 614. Energy harvested from this initial scan also charges the energy storage device 606. In addition, the clock 612 can be dormant when the infusion device is inactive and charging the energy storage device 606 activates and/or sets the clock 612 to the correct time.

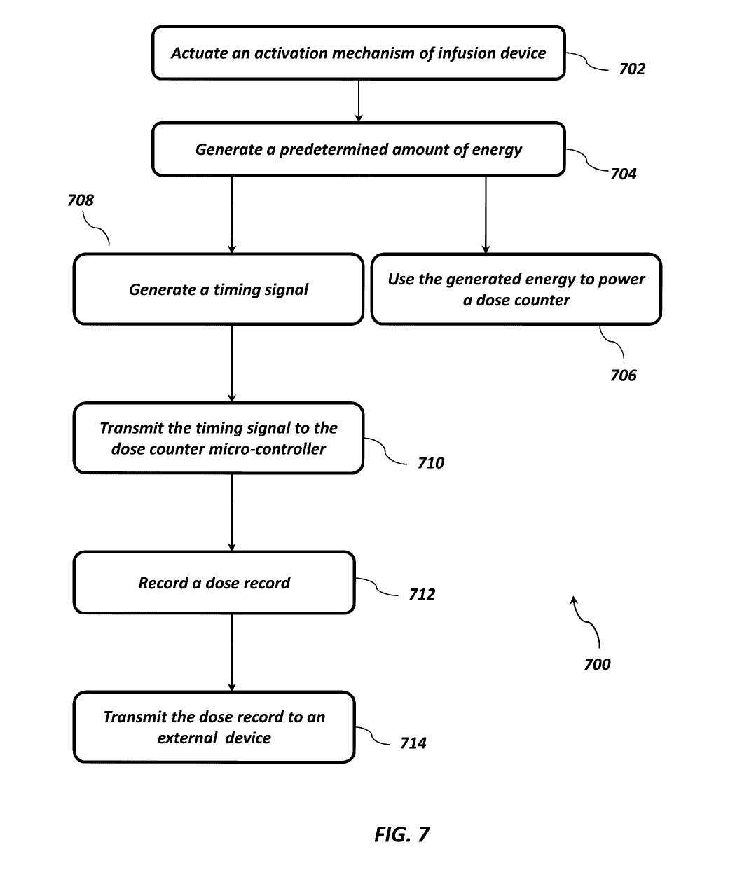

Referring to FIG. 7, an exemplary method 700 of powering a mechanically operated infusion device is described. As described above, the infusion device includes a pump and at least one mechanical activation mechanism for engaging the pump in order to cause a dose event to administer a medicament, such as insulin, to a patient. At block 702, the activation mechanism(s) of the infusion device are activated, such as by depressing the buttons.

At block 704, a predetermined amount of energy is generated by actuation of the activation mechanism(s) of the infusion device. As discussed above, the energy is generated by an energy generator, such as at least one piezo crystal, that is mechanically coupled to the activation mechanism. In this example, actuation of the activation mechanism stresses the piezo crystal coupled to the activation mechanism, generating a piezo energy pulse.

At block 706, the generated energy, e.g., piezo pulse, is supplied to a dose counter in order to power the dose counter. In an example, the generated energy is stored in an energy storage device coupled to the dose counter. In particular, the energy storage device can be coupled to a micro-controller of the dose counter. Energy is transferred from the energy storage device to the micro-controller to power the micro-controller. The energy storage device can be any suitable type of energy storage device, such as at least one capacitor configured for storing the generated energy.

At block 708, a timing signal is generated. In order to create the timing signal, the generated energy is converted, such as by pulse conditioning circuitry, to an electrical signal. The electrical signal is transmitted to the micro-controller of the dose counter. Upon receiving the electrical signal in the micro-controller, a real time clock generates a timing signal that indicates the time at which the energy was generated.

At block 710, the timing signal is transmitted to the micro-controller of the dose counter. At block 712, a dose event is recorded. The dose record includes a timestamp, indicating the time at which the dose event occurred. The timestamp is generated based on the timing signal received in the micro-controller.

At block 714, the dose record is transmitted to an external device, such as a smartphone, via a communication interface. In an example, the micro-controller transfers the dose record each time a record is generated. In another example, the micro-controller can transfer dose records on demand when another device initiates communication with the micro-controller via the communication interface. The communication interface can be any suitable type of communication interface.

As discussed herein, the energy produced by the energy generator(s) powers the components of the infusion device, particularly the components of the dose counter. In an example, the energy generator(s) are piezo crystals coupled to each activation mechanism of the infusion device. Each piezo pulse produced by the piezo crystals may be able to generate energy up to the order of 40 μJ per crystal. In an infusion device including two piezo crystals, approximately 80 μJ is produced per pair of piezo pulses. This energy creates a dose record and maintains the clock. Assuming the micro-controller operates with a core frequency of 1 MHz and 100 clock cycles are required to create a dose record, about 80 μJ is enough energy for the creation of a dose record. The micro-controller may operate at about 1.8V and about 100 μJ for about 100 μs during creation of a dose record, giving a total energy of 18 nJ. Thus, the energy produced by actuating the activation mechanisms is adequate to power the dose counter.

Maintenance of the clock using solely the energy produced by the piezo crystals can be more difficult. Assuming a six hour (21,600 second) runtime, with a 100 nA current drain at approximately 1.8V, the total energy consumed is approximately 3.8 mJ. However, by storing energy produced by the piezo crystals, as well as energy harvested from operation of the communication interface, in an energy storage device, enough energy can be provided to operate the clock. Assuming approximately 1V drop in voltage, the total capacitance required to operate the clock is approximately 2.16 mF, which is achievable by small package capacitors. For example, two approximately 2200 μF capacitors, which retain a charge for twelve hours of clock operation, can provide suitable power to the clock.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “circuit,” “circuitry,” “module,” ‘subsystem” and/or “system.” Furthermore, aspects of the present invention may take the form of a computer program product embodied in one or more computer readable medium(s) having computer readable program code embodied thereon.

### PARTS LIST FOR FIGS. 1-7

10 infusion device

12 enclosure

14 base

16 first activation mechanism

18 second activation mechanism

20 fill port

22 reservoir

24 pump

26 pump piston

28 pump chamber

30 cannula

32 first valve

34 second valve

36 spring

38 spring

40 fluid conduit

42 fluid conduit

44 fluid conduit

46 fluid conduit

48 fluid conduit

50 outlet

52 first linkage

54 second linkage

60 base layer

62 reservoir membrane layer

64 top body layer

66 reservoir portion

68 valve socket

70 valve socket

72 valve seat structure

74 rocker

76 cam cylinder

78 lock tube

80 valve timing cam

82 cam cylinder

84 cam pin

85 fluid Paths

86 planar cap

88 needle

106 energy generator

402 piezo crystal

600 dose counter

602 energy generator

604 converter circuitry

606 energy storage device

608 micro-controller

610 pulse conditioning circuitry

612 real time clock

614 communication interface

616 memory

700 method

702-714 method blocks

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. An infusion device, comprising:
a housing having a reservoir that is sized to retain a quantity of liquid medicament;
a mechanical pump that displaces a portion of the liquid medicament when mechanically actuated;
a mechanically driven activation mechanism disposed on the housing for actuating the pump in order to deliver a dose of liquid medicament and thereby signifying a dose event; and
an energy generator coupled to the activation mechanism, the energy generator being configured to generate energy upon each operation of the mechanically driven activation mechanism in order to power a dose counter that is configured to record dose events.

2. The infusion device of claim 1, wherein the energy generator comprises at least one piezo crystal coupled to the activation mechanism, the at least one piezo crystal being configured to produce a predetermined amount of energy when the activation mechanism is actuated.

3. The infusion device of claim 2, further comprising an energy storage device configured to store energy generated by the energy generator.

4. The infusion device of claim 3, wherein the energy storage device comprises at least one capacitor.

5. The infusion device of claim 3, wherein the dose counter comprises a microcontroller and wherein the energy storage device is coupled to the microcontroller.

6. The infusion device of claim 2, wherein the activation mechanism comprises at least one depressible button coupled to the pump wherein at least one said piezo crystal is coupled to the at least one depressible button.

7. The infusion device of claim 2, in which the activation mechanism is configured to toggle between a non-actuated position and an actuated position and wherein the piezo crystal produces piezo energy pulses upon toggling of the activation mechanism between the non-actuated position and the actuated position.

8. The infusion device of claim 7, wherein the piezo crystal is cantilevered to the activation mechanism such that flexure of the piezo crystal occurs upon toggling of the activation mechanism between the non-actuated position and the actuated position.

9. The infusion device of claim 7, in which piezo energy pulses produced by the flexure of the piezo crystal have opposite polarities based on the toggled position of the activation mechanism.

10. The infusion device of claim 9, further comprising circuitry configured to convert the piezo pulses to a common polarity.

11. The infusion device of claim 2, further comprising circuitry for conditioning the piezo energy pulse produced by the piezo crystal.

12. The infusion device of claim 11, wherein the circuitry further comprises an oscillating crystal that is configured to provide a timing signal based on a generated piezo pulse.

13. The infusion device of claim 12, wherein the dose counter is configured to provide a time stamp of the dose event based on the timing signal.

14. The infusion device of claim 3, further comprising a communication interface for communicating with another device.

15. The infusion device of claim 14, wherein the communication interface comprises a near field communication (NFC) interface.

16. The infusion device of claim 14, wherein operation of the communication interface by said other device generates a predetermined amount of energy and wherein the power source further harvests the generated predetermined amount of energy.

17. The infusion device of claim 16, wherein the harvested energy is stored in the energy storage device and the predetermined amount of energy includes approximately 80 microJoules.

18. The infusion device of claim 16, wherein the harvested energy is used to initiate the dose counter.

19. The infusion device of claim 1, in which the device is configured to administer insulin to a patient.

20. The device of claim 1, wherein the device comprises a portable housing that is configured to be attached directly to skin of a patient.

21. A power source for a medical infusion device, said infusion device comprising a pump and at least one mechanical activation mechanism for engaging the pump to cause a dose event, the power source comprising at least one piezo crystal connected to the activation mechanism and configured to generate a predetermined amount of energy when the device is activated.

22. The power source of claim 21, further comprising a circuit for converting a piezo pulse generated by the at least one piezo crystal to a digital signal.

23. The power source of claim 21, wherein the at least one piezo crystal is cantilevered to the activation mechanism such that flexure of the at least one piezo crystal occurs upon toggling of the activation mechanism between a non-actuated position and an actuated position.

24. The power source of claim 21, wherein the piezo pulses generated by the at least one piezo crystal have opposite polarities based upon the positions of the activation mechanism.

25. The power source of claim 24, further comprising circuitry configured to convert the piezo pulses to a common polarity.

26. The power source of claim 21, wherein the power source is configured to power a dose counter for the medical infusion device.

27. A method for providing electrical power to an infusion device, said infusion device comprising a pump and at least one mechanically operated activation mechanism for engaging the pump to deliver a dose and signify a dose event, the method comprising:
generating a predetermined amount of energy each time the mechanically operated activation mechanism of the infusion device is engaged; and
using the generated energy to power a dose counter of the infusion device.

28. The method of claim 27, wherein the dose counter comprises a micro-controller.

29. The method of claim 28, further comprising the micro-controller recording a dose event when the energy is generated.

30. The method of claim 27, the device further comprising an energy generator for generating the electrical power comprising a piezo crystal coupled to the activation mechanism of the infusion device.

31. The method of claim 27, the device further comprising an energy storage device.

32. The method of claim 31, further comprising storing the energy in the energy storage device and supplying the energy from the energy storage device to the dose counter.

33. The method of claim 27, further comprising converting the energy to a digital signal.
